(19) United States
(12) Patent Application Publication
(10) Pub. No.: US 2006/0084927 A1
(43) Pub. Date: Apr. 20, 2006

(54) Endovascular surgery device

(76) Inventor: Maxime Formichi, Marseille (FR)

Correspondence Address: DENNISON, SCHULTZ, DOUGHERTY & MACDONALD, 1727 KING STREET, SUITE 105, ALEXANDRIA, VA, 22314, US

(21) Appl. No.: 10/536,008

(22) PCT Filed: Nov. 18, 2003
(86) PCT No.: PCT/FR03/03419

§ 371 Date: May 20, 2005

(30) Foreign Application Priority Data
Nov. 28, 2002 (FR) 02/14931

(51) Int. Cl. A61M 5/32

(52) U.S. Cl. 604/272

(57) ABSTRACT

The present invention relates to surgical device enabling a blood vessel to be punctured, in particular an artery, and enabling a radiological guidewire to be inserted in endovascular manner for use in laparoscopic or celioscopic endovascular surgery, in particular of the intra-abdominal blood vessels, the device comprises a first transparent flexible tube co-operating at its distal end with a hollow metal needle to which it is coupled, and in which it is possible to cause a said radiological guidewire to pass, said first tube having, at its proximal end, closure means for closing said first tube, together with insertion means enabling a said radiological guidewire to be inserted in leaktight manner into said first tube.

The present invention relates to a surgical device suitable for endovascular surgery, including interventional radiology.

More particularly, the present invention relates to a device suitable for being implemented by a minimally invasive route, in particular by a laparoscopic and/or celioscopic route, in particular via the large intra-abdominal blood vessels.

### BACKGROUND OF THE INVENTION

At present, endovascular surgery is performed for the following purposes and under the following conditions.

The idea is to insert catheters, in particular balloon catheters and endoprostheses in order to treat arterial aneurysms or stenosis-forming lesions.

These operations are performed either percutaneously, with various possible puncture sites, or by surgical approach via an artery, usually the femoral artery.

Endoprostheses are inserted using catheters inserted inside the blood vessels. Percutaneous insertion can be performed for catheters of small size, in particular of diameter smaller than about 3 millimeters (mm) to 3.60 mm (10 to 12 French).

When an endoprosthesis requires the use of a catheter of larger diameter, it is essential to approach via an artery, and in the great majority of cases, the femoral artery is used.

In practice, the artery is punctured by means of a hollow needle and hemorrhage then occurs in the form of a jet of blood which indicates that the needle is indeed in the arterial lumen, and can be controlled insofar as action is being taken percutaneously, and thus under visual inspection. Then a guide is inserted inside the hollow needle, which guide is known as a “radiological guidewire” and is constituted by a flexible wire having a soft end, making it possible subsequently to insert the catheters required for injecting various substances that are useful for therapeutic or diagnostic purposes, and that are useful above all for inserting a balloon catheter and/or an endoprosthesis.

Surgery via femoral arteries presents certain drawbacks. Firstly, the time required for healing requires at least five days of hospitalization when a surgical opening has been made. Secondly, the size of catheters that can be used remains limited to the size of the femoral artery, i.e. about 7.2 mm to 8.4 mm (24 to 48 French). This is in contrast to certain intra-abdominal vessels of larger diameter that would enable catheters of larger diameter to be inserted.

Finally, some patients have iliac arteries suffering from stenoses, that are tortuous, and/or that are calcified, thus making it difficult or even impossible to advance the catheter inside the artery all the way to the site at which the endoprosthesis is to be delivered.

At present, for percutaneous or femoral approach surgery, hollow needles are used that enable the artery to be punctured and the guidewire to be inserted manually via the needle into the lumen of the artery over a distance that varies depending on the site that is to be reached, and that is at least 20 centimeters (cm) to 30 cm. While the needle is inserted, bleeding occurs. Thereafter, once the guidewire has been put into place, the needle is withdrawn with the guidewire being left in place. Thereafter, said catheter is advanced over said guidewire which is thus located inside the catheter, and thus serves to guide the catheter as far as its end that is located inside the blood vessel.

In order to facilitate subsequent insertion of catheters and endoprostheses, a valve introducer is initially put into place, which introducer consists in a relatively stiff plastic pipe that acts as a protective sheath, and that is surmounted by a leaktight capsule having a flexible membrane that can be perforated by said catheters and that enables the catheters to be inserted in leaktight manner. In general, leaktight capsules also include a lateral opening terminated by a valve or cock that enables various substances to be injected into the blood, and/or that enables the inside of the introducer to be rinsed regularly.

The guidewires in such endovascular procedures continue to be referred to as being “radiological” in spite of the fact that they are also used in operations that are more surgical than radiological, because such guidewires were originally used for positioning catheters or probes radiologically, said catheters or probes serving solely to inject medicinal substances or so-called “contrast” agents used for “arteriography” i.e. radiography of the arteries.

These guidewires are usually made of flexible synthetic material with a resilient core covered in a pliable surface that does not generate thromboses, and that avoids kinking, having a diameter lying in the range 0.35 mm to 0.97 mm (0.014 inches (″) to 0.038″).

Furthermore, laparoscopic or celioscopic approach surgery is known that is performed through the abdominal wall by inserting hollow cylindrical guides referred to as ports having a diameter lying in the range 5 mm to 12 mm, these ports making it possible subsequently to insert surgical instruments and display means such as a camera so as to perform an operation in video-assisted manner inside the abdomen, in particular, and also making it possible to insuflate gas (generally CO₂) into the abdomen in order to enlarge the working space.

However, performing an endovascular procedure following a laparoscopic access, in particular to the intra-abdominal blood vessels, is not possible at present, for the following reasons.

When a large blood vessel inside the abdomen is punctured with a needle, hemorrhage necessarily occurs that might not be controllable as it would be under visual inspection. Such hemorrhage naturally presents a danger to the patient. In addition, it is not possible to suck out the blood since there will be a risk of simultaneously sucking out the gas previously inserted to enable the operation to be performed by the laparoscopic approach after inflating the abdomen. Because blood absorbs light, this makes it difficult or even impossible to view the operation being performed, and thus makes it impossible in practice to perform an endovascular procedure under such conditions.

### OBJECTS AND SUMMARY OF THE INVENTION

The object of the present invention is thus to provide a novel device enabling endovascular procedures to be performed via a laparoscopic approach.

More precisely, an object of the present invention is to provide a device enabling an artery to be punctured while controlling hemorrhaging and while inserting a guidewire into the artery laparoscopically, and subsequently making it possible to perform endovascular insertion of larger-sized catheters and endoprostheses via the laparoscopic approach.

To do this, the present invention provides a surgical device enabling a blood vessel to be punctured, in particular an artery, and a radiological guidewire to be inserted in endovascular manner, for use in laparoscopic or celioscopic endovascular surgery, in particular of the intra-abdominal blood vessels, the device comprising a first transparent flexible tube co-operating at its distal end with a hollow metal needle to which it is coupled, and in which it is possible to cause said radiological guidewire to pass, said first tube having, at its proximal end, closure means for closing said first tube, together with insertion means enabling a said radiological guidewire to be inserted in leaktight manner into said first tube.

It will be understood that the first tube is of dimensions that are sufficient:
to provide the junction between said blood vessel and the surface of the skin with a portion of said first transparent tube extending outside the patient while said vessel is being punctured; and
to contain the jet of blood that escapes from said vessel while it is being punctured.

The device of the present invention enables a blood vessel to be punctured, and in particular a large-diameter artery after a celioscopic access and while avoiding any internal hemorrhage, as would occur with a conventional needle. It is thus particularly useful for puncturing a vessel under celioscopy since the slightest hemorrhage under such circumstances can lead to surgical conversion.

Said first transparent flexible tube acts as a reservoir for blood flowing from the vessel after it has been punctured by said needle without leading to hemorrhage in the field of view of the lens used to visualize the laparoscopic surgery. In addition, and because said tube is transparent, it makes it possible to visualize the color of the liquid contained inside it and thus to recognize backflow of blood and thus check that puncturing has taken place successfully.

Said first transparent tube also makes it possible to convey the radiological guidewire that is to be inserted using the device of the invention so as to take it from the surface of the skin to said needle while said needle is in place in said vessel. The transparency of said first tube also makes it possible to check that radiological guidewires and probes have passed through properly as they progress towards the needle.

It will be understood that the length of said first transparent tube depends on the location of the vessel to be punctured and on the anatomical characteristics of the patient.

Finally, the flexibility of said first transparent tube makes it possible to curve it depending on the orientation of the vessel to be punctured.

The device of the invention also makes it possible, after puncturing, to perform all of the maneuvers required by radiological guidewires, such as insertion and withdrawal from outside the patient towards the lumen of the artery, away from the celioscopic field, i.e. in extra-parietal manner as in a conventional endovascular procedure.

Said first flexible tube also makes it possible to inject substances for diagnostic or therapeutic purposes into the vessels that have been accessed by celioscopy by performing extra-parietal maneuvers away from the celioscopic field.

Once the device has made it possible to perform the maneuvers necessary for launching the endovascular procedure, it can be withdrawn without it being necessary to remove the radiological probes and guidewires inserted into said vessel via the device.

The device of the invention thus makes it possible to develop new surgical techniques associating celioscopy or laparoscopy with endovascular actions, but it can also be used for percutaneous puncturing or puncturing under visual inspection after surgical access.

According to another original and advantageous characteristic of the present invention, said needle presents a longitudinal profile that is curved.

More particularly, the curvature of said needle corresponds to its distal end being inclined relative to its proximal end secured to said coupling element at an angle lying in the range 10° to 45°.

The angle of inclination is measured between the tangents at the proximal and distal ends of said needle.

This curvature of the needle is particularly advantageous in laparoscopic endovascular surgery since it makes it easier to insert the needle into the vessel in such a manner that the bevel tip of the needle lies on the axis of the vessel without puncturing the opposite wall thereof. Whereas in endovascular surgery with percutaneous access the needle is held by the subcutaneous tissue surrounding the vessel, so it is not necessary to enter the needle fully into the vessel, in surgery by a laparoscopic approach, and in particular in intra-abdominal surgery, the vessels are stripped so that the needle is not held by the surrounding tissue, so it is necessary to insert the needle more fully into the vessel without puncturing the opposite wall.

In a particular embodiment, said hollow needle comprises:
a distal portion having a pointed end and including a longitudinal first hollow internal channel, preferably of circular cross-section, through which a said radiological guidewire can be caused to pass; and
a proximal portion forming a coupling element for coupling said needle with said first transparent tube, said coupling element having a second hollow internal channel providing communication between the inside of said first transparent tube and said first hollow channel of said needle, with the internal diameter of said second internal channel of the coupling element being not less than the diameter of said first hollow internal channel.

In an advantageous embodiment, said first transparent tube presents an inside diameter that is greater than or equal to the inside diameter of said second hollow internal channel of said coupling element, which second hollow channel has a transition zone of circular cross-section, that is preferably funnel-shaped, of diameter that decreases progressively until it joins said first hollow internal channel of said needle.

The transition zone inside the coupling element with a progressive change in section serves to direct the radiological guidewire and the radiological probes into the lumen of the needle smoothly and without jerking.

Implementing a first transparent tube of diameter that is relatively large compared with that of the first hollow internal channel inside the needle makes it possible to use a tube having a wall of synthetic material of sufficient thickness to enable said tube to remain both sufficiently flexible and supple to accommodate curving, while avoiding any danger of kinking. Furthermore, it enables a sufficient volume of blood to be contained corresponding to the initial jet of high-pressure blood that escapes from said blood vessel.

Naturally, the size of the outside diameter of said first tube must be smaller than the smallest diameter of the ports put into place during the surgery. In practice, commercially-available surgical ports have an inside diameter that is generally greater than 5 mm, such that a said first transparent tube having an outside diameter of less than 4 mm can be suitable.

Furthermore, the inside diameter of said first tube must be greater than the diameter of radiological guidewires, and preferably greater than the largest-diameter radiological guidewire that is commercially available, i.e. greater than about 1 mm.

In a particular embodiment, said coupling element has at its proximal end a first tubular sleeve with an outside surface onto which the distal end of said first transparent flexible tube is fitted, said first tubular sleeve being extended at its distal end by an intermediate portion providing the junction between said first tubular sleeve and said pointed distal end portion of the needle, such that said intermediate portion presents a circular cross-section of outside diameter greater than or equal to that of the outside diameter of said first tubular sleeve, tapering progressively from its largest-diameter cross-section to its cross-section where it joins said pointed distal end portion of the needle, said second hollow internal channel of said coupling element including, inside said intermediate portion, said funnel-shaped transition zone of cross-section that decreases progressively.

The external profile of said intermediate portion of the coupling element of progressively decreasing cross-section makes it easier to pass said needle and said device as a whole through the laparoscopy or celioscopy port while avoiding jamming at the needle and/or the coupling element, and also avoiding undesirable kinking or twisting in said first transparent tube while passing through said port while said device is being inserted via a laparoscopic port.

According to another advantageous characteristic of the present invention, said first transparent tube has a leaktight capsule at its proximal end for closing said first tube, said capsule having a flexible membrane of incised elastic material suitable for passing a said radiological guidewire therethrough without leaking.

The leaktight capsule acts as a valve preventing any backflow of blood, while allowing radiological guidewires to be inserted without blood leaking out through the capsule. It is possible to use a silicone membrane as said flexible membrane.

Such leaktight capsules are known to the person skilled in the art and commercially available for fitting to the semirigid introducers that are used in endovascular percutaneous surgery.

The term “incised membrane suitable for passing . . . without leaking” is used herein to mean that none of the liquid, in particular blood, contained in said first tube leaks out while said radiological guidewire is being inserted through the incision in the membrane, nor does any leak out after the guidewire has been inserted. It will be understood that the outside surface of the guidewire is wedged in the incision in leaktight manner by the elastic material constituting the membrane.

In a particular embodiment, the membrane is pre-incised with a cross-shaped incision.

In a particular embodiment, said first flexible tube includes injector means enabling liquid to be injected into said first flexible tube, the injector means preferably being constituted by a lateral orifice in the proximal zone of said first tube that is to remain outside the patient, said lateral orifice preferably being integrated in a said leaktight capsule, when present.

Advantageously, said injector means comprises a second flexible tube suitable for fitting to said lateral orifice and including at its free end a cock, and preferably a multi-port cock.

The device of the invention may be presented in the form of a kit comprising various elements such as:
said first transparent flexible tube;
said hollow metal needle;
where appropriate, said closure means, preferably said leaktight capsule; and
where appropriate, said injector means, preferably said second flexible tube, and more preferably including said cock.

These various elements may be preassembled or they may be disassembled, at least in part, in separate packaging, or preferably in common packaging, in particular for subsequent assembly prior to use.

### BRIEF DESCRIPTION OF THE DRAWINGS

Other characteristics and advantages of the present invention appear in the light of the following detailed description made with reference to FIGS. 1 to 4, in which:

FIG. 1 is a diagrammatic view of a device of the invention;

FIG. 2 is a view of a device of the invention having a leaktight capsule 4 provided with a lateral orifice 5;

FIG. 3 is a longitudinal section view of said metal terminal portion including said needle of a device of the invention; and

FIG. 4 is a longitudinal section view of a leaktight capsule 4.

### MORE DETAILED DESCRIPTION

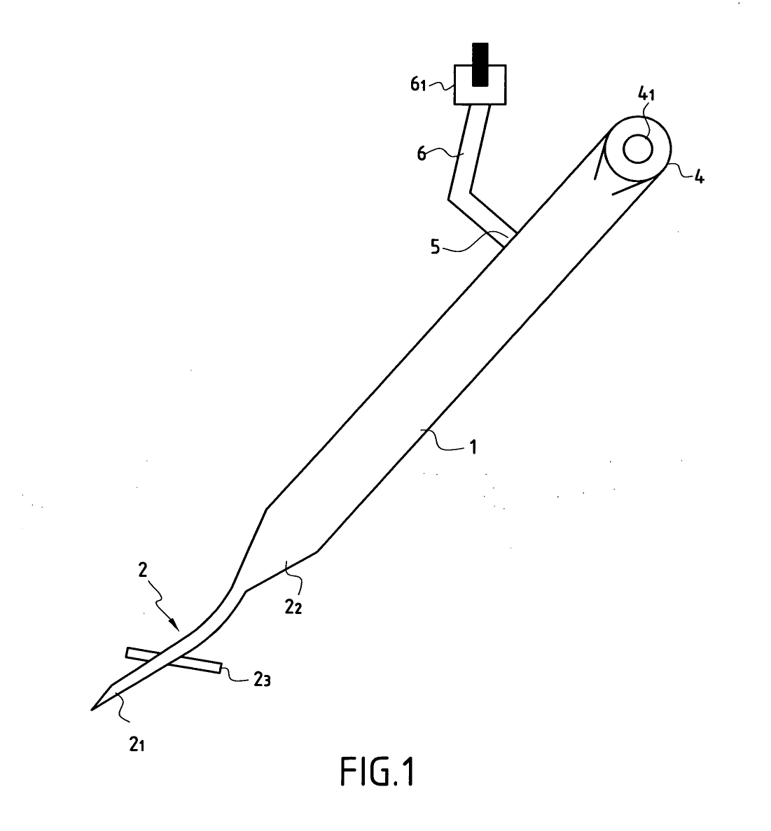

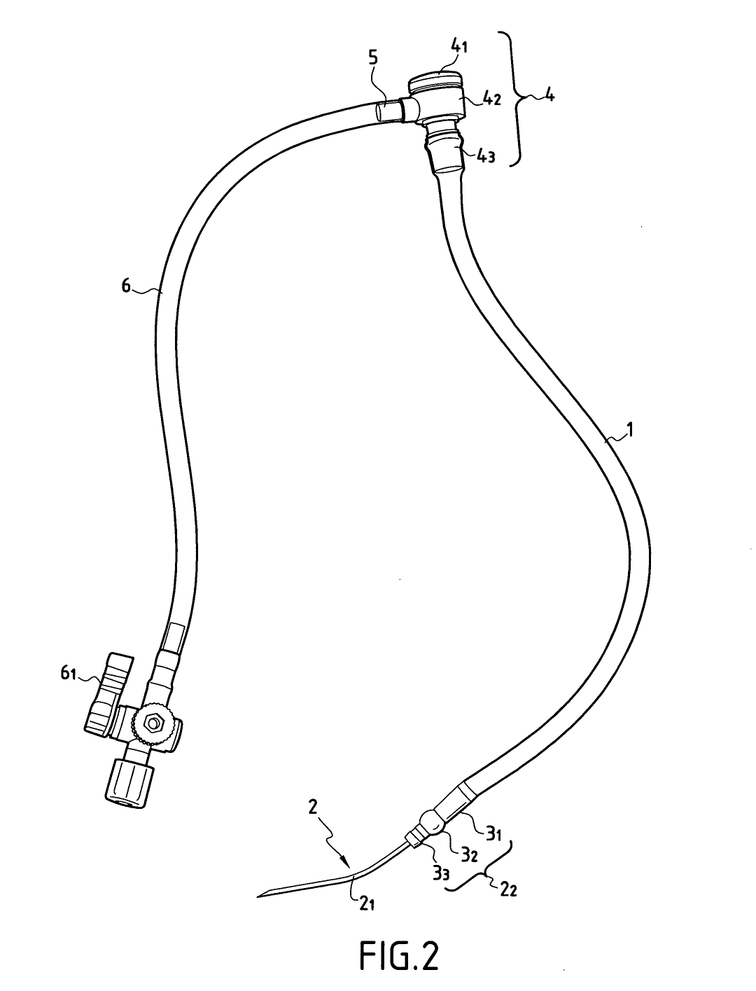

The device of the invention as shown in FIGS. 1 and 2 comprises:
a said first transparent flexible tube 1 which, by way of illustration, presents a length of 20 cm to 50 cm and an outside diameter of 2 mm to 5 mm; and
a hollow needle 2 comprising a curved hollow metal distal portion 2₁ with a first internal channel, and a proximal portion acting as a coupling element 2₂ for coupling with said first transparent tube 1, said coupling element having a second hollow internal channel.

During assembly, The distal end of said first tube 1, which is made of PVC, is engaged as a force-fit on the outside surface of a tubular sleeve 3₁ constituting the proximal portion of said coupling element 2₂.

The coupling element 2₂ has a hollow intermediate portion 3₂ constituted by an enlargement presenting an outline of rounded shape, having an outside diameter greater than the outside diameter of said first tubular sleeve 3₁. Said enlargement 3₂ is situated in line with said first tubular sleeve 3₁, and is made integrally therewith.

Said coupling element 2₂ has a second tubular sleeve 3₃ situated on the side of said enlargement 3₂ that is opposite from said first tubular sleeve 3₁, and is likewise made integrally therewith.

Said needle is made of biocompatible stainless steel.

Said second tubular sleeve 3₃ serves firstly to provide a junction between the pointed distal portion 2₁ of the hollow needle 2 and the coupling element 2₂, and secondly it enables a small plastic flexible tube (not shown) to be fitted thereon to cover the pointed distal portion 2₁ of the hollow needle 2, so as to protect it prior to use in order to avoid jabs that could spoil its conditioning or injure personnel handling it prior to the intervention.

The dimensions of the needle 2 are adapted as a function of the size of the vessel to be punctured and the size of the radiology guidewires that are to be inserted subsequently.

The size of the radiology guidewires depends mainly on the locations of the vessels in the body and on the catheters that are to be inserted subsequently using said guidewires, which also depend on the size of the endoprosthesis or other object that is to be inserted subsequently in endovascular manner.

By way of illustration, in practice, needles 2 are used having the following dimensions:
length 2 cm to 5 cm;
outside diameter 0.5 mm to 3 mm;
including a bend corresponding to an angle of inclination in the range 20° to 30°0; and
said first hollow internal channel has a diameter of 0.35 mm to 2 mm.

The dimensions of said coupling element 2₂ are adapted as a function of the inside diameter of said first tube 1, which depends on the length and thus on the morphology of the patient to be operated.

Said first tubular sleeve 3₁ presents an outside diameter that is substantially identical to the inside diameter of said first transparent tube 1. In practice, and by way of illustration, said first tubular sleeve 3₁ has a length lying in the range 5 mm to 10 mm for a said first transparent tube having an outside diameter lying in the range 3 mm to 5 mm.

Said coupling element 2₂ has a second hollow internal channel beginning at the inside of said first tubular sleeve 3₁, passing through the inside of said enlargement 3₂, and terminating via the inside of said second tubular sleeve 3₃.

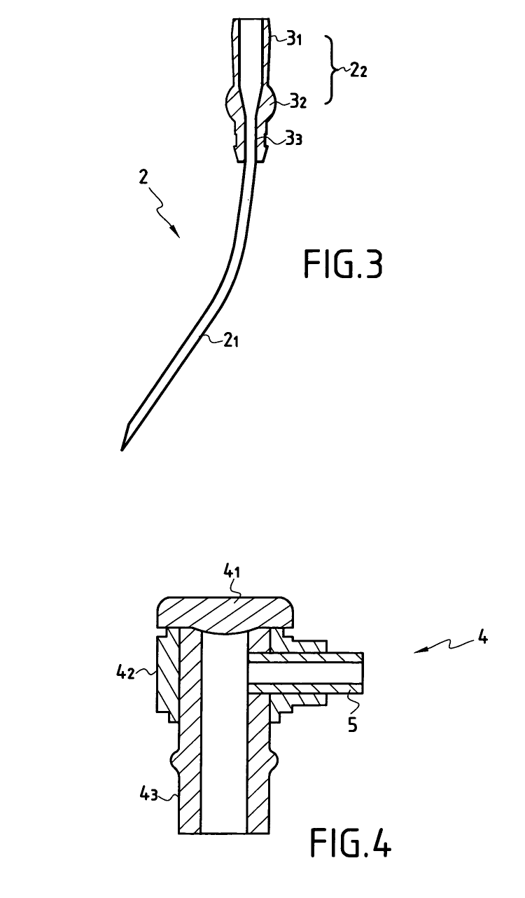

As can be seen in FIG. 3, the longitudinal section of said needle 2 shows that said second internal channel forms a funnel with its diameter decreasing progressively from the distal end of said first tubular sleeve 3₁ to the distal end of said second tubular sleeve 3₃, which sleeve is extended by the proximal end of said first internal channel of the pointed distal portion 2₁ of the needle.

The proximal end of said first tube 1 is assembled to a leaktight capsule 4. This leaktight capsule 4 has a substantially cylindrical central compartment 4₂ with a top orifice that is covered by a resilient flexible membrane 4₁ including a leaktight incision in the form of a cross, i.e. the material from which said membrane is made is sufficiently flexible and strong to ensure firstly that there is no leakage of liquid blood back through the incision, and secondly to enable the incision to allow radiology guidewires having a diameter of 0.35 mm to 2 mm to be inserted without the liquid contained in said first tube leaking out at the junction between said radiology guidewire and the membrane 4₁. Said central compartment 4₂ is extended at its distal end by a third tubular sleeve 4₃ that has the proximal end of said first transparent tube fitted onto the outside face thereof. The central compartment 4₂ further includes a lateral orifice 5 in the form of a fourth tubular sleeve onto which there is fitted a second transparent flexible tube 6, itself having its opposite end assembled to a multi-port cock 6.

Leaktight capsules 4 as described above are marketed in particular by the Japanese Terumo Corporation under the trademark Radiofocus®.

Said second tube 6 thus enables a liquid containing substances for diagnostic or therapeutic purposes to be injected into the inside of said first transparent tube and thus into the inside of said vessel, or indeed it enables a rinsing liquid to be injected into said first tube to prevent the blood it contains from coagulating, in particular a liquid with heparinized serum.

Said cock 6₁ serves to close said second tube 6 and thus also said first tube 1 to which it is connected. Advantageously, it has a plurality of insertion ports, e.g. to make it possible to track blood pressure measurements on one port and to inject said substances for diagnostic or therapeutic purposes via another port.

FIG. 1 shows an abutment 2₃ level with the hollow needle 2₁ serving to prevent the needle being pushed in too far.

In FIG. 2, the intermediate portion having a rounded outline of the coupling element constitutes an enlargement 3₂.

The device of the invention, as described above, has been used to implant Talent® endoprostheses from Metronic Ave (USA), measuring 12 mm to 20 mm in diameter and 95 mm to 110 mm in length, once deployed in blood vessels.

The catheters containing the endoprostheses were about 5.4 mm in size (18 French).

Eight agricultural pigs were selected for the experiment and treated in application of a protocol complying with laboratory animal care legislation.

The animal was placed in a right lateral decubitus position with a block elevating the thoraco-abdominal junction. The operators were positioned on the ventral side, with the video column facing the dorsal side.

The infra-renal abdominal aorta was engaged by the retroperitoneal laparoscopic route after placing three 10 mm ports in the left flank between the iliac crest and the 11th rib. The retroperitoneum was maintained at a pressure of 12 millimeters of mercury (mmHg) throughout the operation. The segment of artery between the left renal artery and the aortic trifurcation was dissected, being secured by two gauze straps with transparietal pull-cords to enable upstream and downstream clamping to be performed at any instant during the endovascular time. The visible arteries were clipped to limit bleeding.

A 180 cm long 0.89 mm (0.035 inch) diameter Terumo® guidewire was introduced into the retro-peritoneal space via the ports and then under laparoscopic inspection into the aorta after direct needle puncture into the aorta without clamping. The guidewire was thus positioned approximately 60 cm upstream inside the thoracic aorta.

Hemostasis around the guidewire was maintained after it had been withdrawn by a single clamp holding the aortic wall. An IV dose of heparin was injected from the cock 6₁.

An approximately 5.4 mm catheter (18 French) containing an endoprosthesis was inserted on the guidewire via the same port initially into the retro-peritoneal space, and then into the infra-renal aorta by progressively widening the puncture orifice without clamping. The length of the catheters was determined so as to reach the descending thoracic aorta without any radiological monitoring and so as to enable the endoprosthesis to be deployed between the left subclavian artery and the celiac trunk. Release was performed conventionally by withdrawing the outer sheath of the catheter. To extract the catheter from the abdominal aorta, clamping upstream and downstream of the insertion orifice using the pull-cord gauze straps enables bleeding to be controlled. The aorta was closed either by means of a pursestring suture made before needle puncture, or else by direct suturing after the catheter had been withdrawn.

After arterial circulation had been reestablished and the aorta had been checked to make sure there was no leakage, the laparoscopy ports were removed, and the orifices through the skin were closed.

The quality of revascularization downstream from the procedure was evaluated by the return to normal of the oxymetric curve recorded in the tail region of the animal.

The pigs were subsequently euthanasized, and the positioning and the permeability of the endoprosthesis were verified visually.

The operation was achieved successfully with seven animals using the established protocol. For those seven animals, blood losses were well controlled. Mean bleeding was 120 milliliters (mL), lying in the range 50 mL to 210 mL. The blood losses recorded at the time of needle puncture were negligible. The insertion of the guidewires and the various catheters did not lead to additional bleeding.

In six animals, the retro-peritoneal laparoscopic approach gave an excellent view of the infra-renal aorta. The aorta detection time was long, lying in the range 92 minutes (min) to 233 min.

In two cases, there was accidental opening of the peritoneum. It was necessary to put a needle into place through the abdominal wall to deflate the peritoneal cavity. This complication increased the total operating time to 240 min and 300 min, respectively.

On two occasions it was difficult to insert the approximately 5.4 mm (18 French) catheter into the aorta. It was necessary to widen the entry orifice with laparoscopic scissors after temporary clamping of the abdominal aorta.

The aortic access orifice remains leaktight without additional means being required while the approximately 5.4 mm (18 French) catheter was in the aorta. After the catheter had been withdrawn, bleeding in the aortic insertion site was controlled by the proximal and distal clamps and the prior ligation of the lumbar arteries. The endovascular time from puncturing the artery to withdrawing the catheter was short. It was 22 min on average (in the range 10 min to 35 min). The mean clamping time needed for closing the aortic orifice was 30 min on average, lying in the range 15 min to 70 min.

The total duration of the operation was 205 min on average. No significant systemic hemodynamic trouble occurred during the operation. Harvested thoracic aortic segments showed that the device was properly positioned and deployed in all cases.

The use of a laparocscopic approach route from abdominal blood vessels would appear to be of advantage for patients having femoral and iliac arteries in poor state. Arterial pathology at this location can be a contraindication for implanting an aortic endoprosthesis for aneurysm.

For thoracic endovascular procedures, a laparoscopic route enables the distance between the point of entry into the arterial system and the release point to be shortened. This reduces stresses due to friction and also makes it possible to use catheters of larger diameter. In addition, a reduction in hospitalization time can be expected because of the faster healing of a laparoscopic approach compared with the usual surgical approach via the femur.

1. A surgical device enabling a blood vessel to be punctured, in particular an artery, and a radiological guidewire to be inserted in endovascular manner, for use in laparoscopic or celioscopic endovascular surgery, in particular of the intra-abdominal blood vessels, the device being characterized in that it comprises a first transparent flexible tube (1) co-operating at its distal end with a hollow metal needle (2) to which it is coupled, and in which it is possible to cause said radiological guidewire to pass, said first tube having, at its proximal end, closure means (4) for closing said first tube, together with insertion means (4₁) enabling a said radiological guidewire to be inserted in leaktight manner into said first tube.

2. A device according to claim 1, characterized in that said needle (2) presents a curved longitudinal profile.

3. A device according to claim 2, characterized in that the curvature of said needle (2) corresponds to its distal end being inclined relative to its proximal end secured to said coupling element (2₂) by an angle lying in the range 10° to 45°.

4. A device according to claim 1, characterized in that said hollow metal needle (2) comprises:
a distal portion (2₁) with a pointed end and having a first hollow longitudinal internal channel, preferably of circular cross-section, through which a said radiological guidewire can be inserted; and
a proximal portion forming a coupling element (2₂) for coupling said needle (2) to said first transparent tube (1), said coupling element (2₂) having a second hollow internal channel providing communication between the inside of said first transparent tube (1) and said first hollow channel of said needle, and the inside diameter of said second internal channel of the coupling element (2₂) being not less than the diameter of said first hollow internal channel.

5. A device according to claim 4, characterized in that said first transparent tube (1) presents an inside diameter that is greater than or equal to the inside diameter of said second hollow internal channel of said coupling element (2₂), which second hollow channel includes a transition zone of circular cross-section in the form of a funnel of diameter that decreases progressively until it joins said first hollow internal channel of said needle (2₁).

6. A device according to claim 5, characterized in that said coupling element (2₂) comprises at its proximal end a first tubular sleeve (3₁) having an outside surface onto which the distal end of said first transparent tube (1) is fitted, said first sleeve (3₁) being extended at the distal end by an intermediate portion (3₂) providing the junction between said first tubular sleeve (3₁) and said pointed distal portion (2₁) of the needle, such that said intermediate portion (3₂) presents a circular cross-section of outside diameter greater than or equal to that of the outside diameter of said first tubular sleeve, and decreases progressively from its largest diameter cross-section to its cross-section at the junction with said distal portion at the pointed end (2₁) of the needle, said second hollow internal channel of said coupling element (2₂) including inside said intermediate portion (3₂) said funnel-shaped transition zone of cross-section that decreases progressively.

7. A device according to claim 1, characterized in that said first transparent tube (1) includes a leaktight capsule (4) at its proximal end for closing said first tube (1), said capsule (4) including a flexible membrane (4₁) of elastic material, the membrane being incised so as to be suitable for having a said radiological guidewire pass therethrough in leaktight manner.

8. A device according to claim 1, characterized in that said first tube (1) includes injection means (5) enabling a liquid to be injected into said first tube (1), the injection means preferably being constituted by a lateral orifice (5) in the proximal zone of said first tube that is to remain outside said patient, said lateral orifice (5) preferably being integrated in a said leaktight capsule (4), when present.

9. A device according to claim 8, characterized in that said injection means (5) comprises a second flexible tube (6) suitable for fitting to said lateral orifice (5) and having at its free end a cock (61), preferably a multi-port cock.

10. A kit of elements suitable for being assembled together to provide a device according to claim 1, the kit being characterized in that said elements comprise:
said first transparent flexible tube (1);
said hollow metal needle (2);
where appropriate, said closure means, preferably said leaktight capsule (4); and
where appropriate, said injection means, preferably said second flexible tube (6) and more preferably including said cock (6₁);
said elements being preassembled or dissembled, at least in part, and preferably in the same packaging.
